(19) United States
(12) Patent Application Publication
(10) Pub. No.: US 2005/0070970 A1
(43) Pub. Date: Mar. 31, 2005

(54) Movement disorder stimulation with neural block

(76) Inventors: Mark B. Knudson, Shoreview, MN (US); Richard R. Wilson, Arden Hills, MN (US); Katherine S. Tweden, Mahtomedi, MN (US); Timothy R. Conrad, Eden Prairie, MN (US)

Correspondence Address: MERCHANT & GOULD PC, P.O. BOX 2903, MINNEAPOLIS, MN, 55402-0903, US

(21) Appl. No.: 10/756,166

(22) Filed: Jan. 12, 2004

Related U.S. Application Data
(63) Continuation-in-part of application No. 10/674,330, filed on Sep. 29, 2003.
(63) Continuation-in-part of application No. 10/675,818, filed on Sep. 29, 2003.
(63) Continuation-in-part of application No. 10/674,324, filed on Sep. 29, 2003.
(63) Continuation-in-part of application No. 10/752,944, filed on Jan. 6, 2004.
(63) Continuation-in-part of application No. 10/752,940, filed on Jan. 6, 2004.

(51) Int. Cl. A61N 1/18

(52) U.S. Cl. 607/45

(57) ABSTRACT

A method and apparatus for treating patients suffering from involuntary movement disorders (including epilepsy) by stimulating a selected cranial nerve of the patient with an electrical signal applied to induce a signal at brain by applying an electrical signal at the nerve to ameliorate the disorder and by applying a neural conduction block at the nerve selected to at least partially block nerve impulses on said nerve at a blocking site and reduce adverse effects of said signal on an organ.

### I. CROSS-REFERENCE TO RELATED APPLICATIONS

This application is a continuation-in-part of the following U.S. patent applications, each filed Sep. 29, 2003: Ser. No. 10/674,330 titled “Nerve Conduction Block Treatment”; Ser. No. 10/675,818 titled “Enteric Rhythm Management” and Ser. No. 10/674,324 titled “Nerve Stimulation And Conduction Block Therapy”. The present application is also a continuation-in-part of U.S. Ser. No. [not yet assigned], attorney docket number 14283.1USI4 titled “Electrode Band Apparatus and Method” and U.S. Ser. No. [not yet assigned], attorney docket number 14283.1USI5 titled “Intraluminal Electrode Apparatus and Method”, each filed Jan. 6, 2004 in the names of the same inventors as in the present application.

### II. BACKGROUND OF THE INVENTION

1. Field of the Invention

The present invention is directed toward a method and apparatus for alleviating or preventing epileptic seizures and other clinical conditions of the nervous system including movement disorders. More particularly, the present invention is directed to an improvement in such devices and related methods of use in a manner to reduce a likelihood of adverse side effects and enhance the usability of such devices and methods.

2. Description of the Prior Art

A prior art device and method of use of vagal stimulation to treat epileptic seizures or other clinical conditions of the nervous system are described in U.S. Pat. No. 4,702,254 to Zabara dated Oct. 27, 1987; U.S. Pat. No. 4,867,164 to Zabara dated Sep. 19, 1989 and U.S. Pat. No. 5,025,807 to Zabara dated Jun. 25, 1991 (all incorporated herein by reference and respectively referred to herein as the “'254 patent”, the “'164 patent” and the “'807 patent”). A prior art device and method of use of near-diaphragmatic nerve stimulation to treat movement disorders are described in U.S. Pat. No. 6,622,038 to Barret et al., dated Sep. 16, 2003 (incorporated herein by reference and referred to herein as the “'038 patent”).

A problem associated with nerve stimulation is the creation of undesired side effects. For example, stimulation of the vagus nerve in the neck can create undesired cardiac or voice responses. Stimulation near a diaphragm can have cardiopulmonary effect as well as undesired gastrointestinal effects or pancreobiliary effects. Another potential problem associated with nerve stimulation is that antidromic inhibitory responses may interfere with the effectiveness of the procedure.

U.S. Pat. No. 5,205,285 to Baker, Jr. dated Apr. 27, 1993 describes voice suppression of vagal stimulation as an attempt to address the issue of unwanted side effects. The '285 patent states that in at least some patients receiving vagal stimulation treatment for epileptic seizures, there is a noticeable modulation of speech during actual application of the stimulation. According to the teachings of U.S. Pat. No. 5,205,285 (incorporated herein by reference), the vagal stimulation for seizure treatment is de-activated during periods of speech.

Unwanted side effects can also be addressed by lowering the energy levels of stimulation or reducing the duration over which stimulation therapy is applied. Both of these reduce the efficacy of treatment.

Another technique for addressing the side effects is to permit a patient to control when a stimulation is applied. A patient activation of stimulation therapy is described in U.S. Pat. No. 5,304,206 to Baker Jr., et al. dated Apr. 19, 1994. Again, by the time a patient senses a need for therapy, the ability to effectively intervene may be compromised. Furthermore, patient control is unreliable.

An object of the present invention is to provide a neural conduction block to the vagas in combination with stimulation to block signals at the blocking site. The present invention describes a blocking of a nerve (such as the vagal nerve) to avoid antidromic influences during stimulation or to block stimulation signals which might otherwise result in adverse side effects. Cryogenic nerve blocking of the vagus is described in Dapoigny et al., “Vagal influence on colonic motor activity in conscious nonhuman primates”, Am. J. Physiol., 262: G231-G236 (1992). Electrically induced nerve blocking is described in Van Den Honert, et al., “Generation of Unidirectionally Propagated Action Potentials in a Peripheral Nerve by Brief Stimuli”, Science, Vol. 206, pp. 1311-1312. An electrical nerve block is described in Solomonow, et al., “Control of Muscle Contractile Force through Indirect High-Frequency Stimulation”, Am. J. of Physical Medicine, Vol. 62, No. 2, pp. 71-82 (1983) and Petrofsky, et al., “Impact of Recruitment Order on Electrode Design for Neural_Prosthetics of Skeletal Muscle”, Am. J. of Physical Medicine, Vol. 60, No. 5, pp. 243-253 (1981). A neural prosthesis with an electrical nerve block is also described in U.S. Patent Application Publication No. US 2002/0055779 A1 to Andrews published May 9, 2002. A cryogenic vagal block and resulting effect on gastric emptying are described in Paterson Calif., et al., “Determinants of Occurrence and Volume of Transpyloric Flow During Gastric Emptying of Liquids in Dogs: Importance of Vagal Input”, Dig Dis Sci, (2000);45:1509-1516.

### III. SUMMARY OF THE INVENTION

According to a preferred embodiment of the present invention, a method and apparatus are disclosed for treating patients suffering from involuntary movement disorders (including epilepsy) by stimulating a selected cranial nerve of the patient with an electrical signal applied to induce a signal at the brain by applying an electrical signal at the nerve to ameliorate the disorder and by applying a neural conduction block at the nerve selected to at least partially block nerve impulses on said nerve at a blocking site and reduce adverse effects of the electrical signal on an organ.

### IV. BRIEF DESCRIPTION OF THE DRAWINGS

FIG. 1 is a schematic representation of a totally implanted neurocybernetic prosthesis constructed in accordance with the principles of the prior art as described in U.S. Pat. No. 4,702,254 present invention and showing the manner in which the same is tuned;

FIG. 2 is a schematic representation of a partially implanted neurocybernetic prosthesis according to the afore-said prior art;

FIG. 3 is a schematic representation of a sensor-feed-back system for automatically initiating the neurocybernetic prosthesis according to the afore-said prior art;

FIG. 4 schematically illustrates the placement of an electrode patch on the vagus nerve and the relationship of the vagus nerve with adjacent structures according to the afore-said prior art, and

FIG. 5 schematically represents the preferred placement of the pulse generator and electrode patch of the present invention in the human body according to the afore-said prior art;

FIG. 6 is view similar to FIG. 4 showing a blocking electrode according to the present invention positioned on the cardiac nerve with the blocking electrode positioned between the heart and the electrode patch;

FIG. 7 is view similar to FIG. 4 showing a blocking electrode according to the present invention positioned on the nerve from the vagus to the vocal cords of the patient with the blocking electrode positioned between the vocal cords and the electrode patch;

FIG. 8 is view similar to FIG. 5 showing a blocking electrode according to the present invention positioned on the vagus nerve distal to the electrode patch with the blocking electrode positioned between the electrode patch and distal organs (such as cardiopulmonary and gastrointestinal organs);

FIG. 9 is a prior art representation from U.S. Pat. No. 6,622,038 showing a simplified partial front view of a patient (in phantom) having an implanted neurostimulator for generating the desired signal stimuli which are applied directly and bilaterally at a near-diaphragmatic location to the right and left branches of the patient's vagus via an implanted lead/nerve electrode system electrically connected to the neurostimulator;

FIG. 10 is a representation of the afore-said prior art showing a simplified partial front view of a patient similar to that of FIG. 9, but in which a pair of implanted neurostimulators is used for generating the desired signal stimuli;

FIG. 11 is a representation of the afore-said prior art showing a simplified partial front view of a patient in which an implanted neurostimulator and associated electrode is used for unilateral stimulation of only one branch of the vagus nerve at the near-diaphragmatic location;

FIG. 12 is a representation of the afore-said prior art showing a simplified partial front view of a patient in which the signal stimuli are applied at a portion of the nervous system remote from the vagus nerve, for indirect stimulation of the vagus nerve at the near-diaphragmatic location;

FIG. 13 is the view of FIG. 9 modified according to the teachings of the present invention;

FIG. 14 is the view of FIG. 10 modified according to the teachings of the present invention;

FIG. 15 is the view of FIG. 11 modified according to the teachings of the present invention; and

FIG. 16 is the view of FIG. 12 modified according to the teachings of the present invention.

### V. DESCRIPTION OF THE INVENTION

Referring now to the several drawing figures in which identical elements are numbered identically throughout, a description of a preferred embodiment of the present invention will now be provided. For ease of understanding, a description of the prior art as appears in prior art patents will first be provided following by a description of the present invention. In the sections of this application pertaining to teachings of the prior art, the specification from prior art patents is substantially reproduced for ease of understanding the embodiment of the present invention. For the purpose of the present application, Applicants accept the accuracy of information in those patents without independent verification.

A. Teachings of Prior Art for Near-Cranial Application

For ease of illustrating the present invention in a preferred embodiment for treating epileptic seizures and other clinical conditions of the nervous system, the description of the invention of U.S. Pat. No. 4,702,254 to Zabara dated Oct. 27, 1987, U.S. Pat. No. 4,867,164 to Zabara dated Sep. 19, 1989 and U.S. Pat. No. 5,025,807 to Zabara dated Jun. 25, 1991 (respectively, the '254 patent, the '164 patent and the '807 patent and all incorporated herein by reference) is presented in this section of this application (collectively the “Zabara patents”).

The invention of the Zabara patents purports to operate utilizing a principle called neurocybernetic spectral discrimination and works in the following way. Since, in general, nerves are of a microscopic diameter and are combined together in a nonhomogeneous mixture of diameters and functional properties, it is not presently possible to adequately control external current to selectively activate a specific group of nerves embedded within a relatively large number of other nerves. Spectral discrimination acts to overcome this fundamental problem by “tuning” the external current (electrical generator) to the electrochemical properties of the selected nerves.

The electrochemical properties utilized in the design of the discriminator are: action potential, conduction velocity, refractory period, threshold, resting membrane potential and synaptic transmission. In addition, there are two general properties of the brain called central excitatory state and hypersynchronicity which can be explained in the following manner.

All nerves can be divided into two functional types: excitatory and inhibitory. The spectral discriminator acts to selectively activate those inhibitory nerves which can prevent or block the epileptic seizure. In other words, these specific inhibitory nerves are embedded in a bundle or cable of nerve fibers of varied functions and properties. A bundle of such nerves may typically consist of 100,000 or more individual fibers and contain mixed excitatory and inhibitory characteristics. The purposeful design of the discriminator is to activate just those relatively few nerves which are inhibitory to the epileptic seizure.

Thus, it must be possible to “discriminate” those desired fibers within a broad spectrum of nerves. One reason that this is important is that if excitatory fibers are simultaneously activated with inhibitory fibers then the desired effect of inhibition on the seizure may be nullified. There is a balance of excitation and inhibition in the brain called the central excitatory state which is affected by specific electrochemical signals. Epilepsy is the increase of the central excitatory state to an abnormal level as based on a hypersynchronous discharge of neurons. A second reason for spectral discrimination is to prevent undesirable side effects by activating other nerves unnecessarily.

There is a physiological basis for the effectiveness of the selected nerves in blocking or preventing epileptic seizures. The activation of these nerves produces an effect on the reticular system via synaptic transmission. The reticular system has been demonstrated to be important in whatever abnormality leads to epileptic seizures. The reticular system is a relatively large and inhomogeneously constituted structure extending from the hind-brain (medulla) to the mid-brain (thalamus) with neural connections to the cerebral cortex and spinal cord. It is not practical at present to directly electrically activate the reticular system because of its large extent and proximity to vital centers. Thus, it was important to discover what nerves might innervate the reticular system sufficiently to produce a significant effect on the reticular system; the net effect being to produce inhibition of epileptic seizures.

For the purpose of interfacing the prosthesis with the critical processes of the brain, inhibition can also be called by its comparable engineering term of negative feedback. Further, it is possible that the seizure originates due to a temporary lack of diminution of negative feedback from the reticular system to seizure sites in the brain. By acting on appropriately selected nerves, the prosthesis results in the replacement of this negative feedback and thus prevents the seizure.

The approach of spectral discrimination is to utilize the basic properties of conduction velocity, diameter, refractory period, threshold, membrane potential, action potential, after potentials, synchronization and synaptic transmission. Based on these properties, the parameters of the pulse generator are chosen in terms of frequency, duration of pulse wave, shape of wave, voltage or current and duration of pulse train. In addition, a time dependent direct current polarization of the membrane can be utilized to produce a “gate” effect.

The “gate” effect is based upon the polarization characteristics of the neural membrane. The membrane potential across the neural membrane can be increased to a point where a block of conduction results. It is a method of separating relatively slower conducting fibers from faster conducting fibers. For example, when the nerve is activated, the action potentials of higher velocity (A) will lead the slower ones (C). A “polarization” block on the nerve membrane will stop A and then the block is removed before C arrives so that the net result is that A, but not C, is prevented from continuing.

The next step is to determine the locus of action of the current generated by the spectral discriminator. This problem relates to the important area of interface between the electronic pulse generator and control signal generated within the brain. In addition, this interface should be of such a nature that the pulse generator is located external to the brain but at the same time the current be set in a compact and identifiable region of nerves so that the site of current is specific and reproducible from patient to patient; no cell bodies are located within the targeted area for current (due to possible production of cell deterioration by the current); and the nerves produce the desired effect on brain operations via sites of synaptic connection.

Analysis by spectral discrimination has demonstrated that the most desirable extra-cranial sites for all these effects are the cranial nerves. Specific cranial nerves have been determined to be optimum for beneficial effects on neurological problems. In particular, the vagus nerve is the optimum site for control of epileptic seizures. If the total spectrum of the nerve is not known, it is possible to activate all the nerve fibers by the spectral discriminator and record the response on an oscilloscope. From this total fiber spectrum, it is possible to determine the settings of the spectral discriminator to select the activation of the appropriate subset of nerves.

Thus, it is possible to identify by the operation of the spectral discriminator those nerves which can produce the desired corrective signal. Spectral discrimination is not only a therapeutic prosthesis method but it is also the method of analysis to determine nervous system sites for beneficial effects in neurological problems.

In one form of the invention of the Zabara patents, the neurocybernetic prosthesis need be turned on only during the duration of a seizure. It can be turned on either manually (by the patient) or automatically by a sensor-feedback system. Many epileptics have sensory signs immediately preceding the convulsion called an aura. At the initiation of the aura, the patient will be able to turn on the device and prevent the seizure. On the other hand, the neurocybernetic prosthesis can include a sensor-feedback system to block the seizure automatically. This feedback system would include sensors specifically designed to determine relatively instantaneous changes in the values of state parameters, which precede eruption of the hypersynchronous activity. Such parameters might include electroencephalographic waves, respiration changes, heart rate changes, various auras or motor effects such as ties or myoclonic jerks. The prosthesis thereby can be activated by sensor feedback producing a signal which precedes convulsive hypersynchronous discharge.

According to the Zabara patents, it is also believed that the neurocybernetic prosthesis can be used prophylactically. That is, the prosthesis could be activated periodically whether or not an aura or other condition is sensed. Preferably, during a treatment period, the prosthesis may be activated once every hour or so for a minute or more with the frequency and duration gradually reduced to nothing at the end of the period which may be a week or more. It is believed by Zabara that such treatment may eliminate seizures or reduce their frequency and intensity. This continuous cycling on and off is also believed by Zabara to be most useful for treating continuous or chronic tremors such as Parkinsonism.

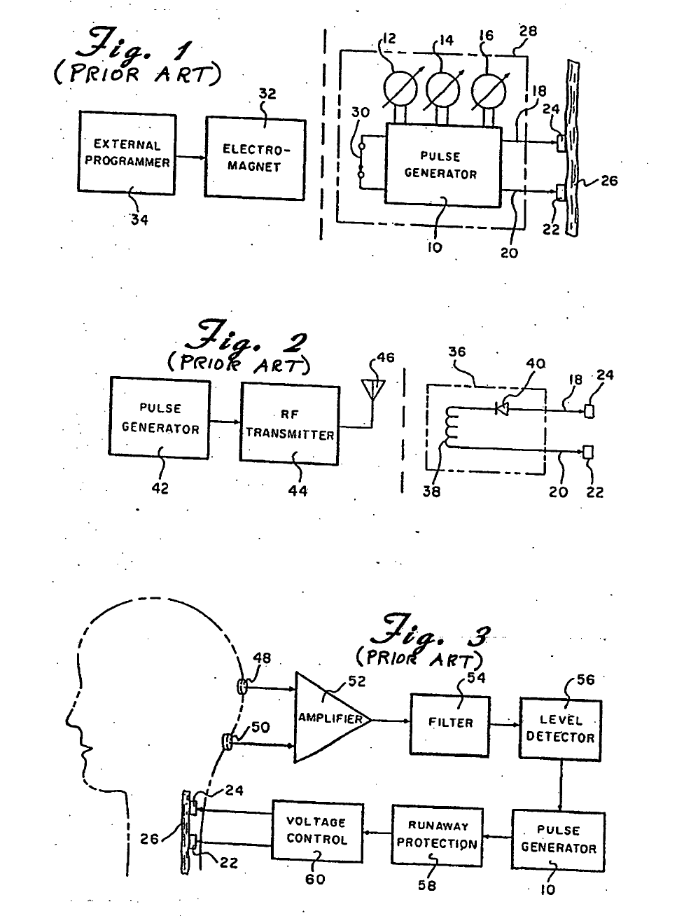

One example of an electrical circuit for practicing the present invention is shown schematically in FIG. 1. The circuit is comprised essentially of a pulse generator 10 which is capable of generating electrical pulses having a frequency of between 30 and 300 cycles per second, a pulse duration of between 0.3 and 1 millisecond and a constant current of between approximately 1 and 20 milliamperes. The frequency, pulse width and the voltage or current level of the output signal form the pulse generator can be varied by controls 12, 14 and 16. Although the pulse width and current or voltage are set by the controls 14 and 16, it is preferred that the generator 10 be of the type which is capable of ramping up to the set pulse width and/or current or voltage whenever the generator is activated. This technique is to eliminate involuntary twitching when the prosthesis is activated and is particularly useful when continuous types of tremors are being controlled or suppressed by the prosthesis. Electrode leads 18 and 20 are connected to electrodes 22 and 24 which are applied to the vagus nerve 26 in a manner to be more fully described hereinafter.

In the preferred embodiment of the Zabara patents, the pulse generator 10 with its battery pack and other associated circuits are preferably intended to be fully implanted. For this reason, the generator is enclosed in an epoxy-titanium shell 28 (or similar bio-compatible material). As described above, the present invention operates utilizing the principle of neurocybernetic spectral discrimination. The prosthesis must, therefore, combine the desired current parameters to correspond to the specific properties (linear and non-linear) of the selected nerves. Thus, the command signal of the device is a function of the following specific nerve properties: refractory periods, conduction velocity, synchronization or de-synchronization, threshold and brain inhibitory state. In a sense, the current parameters must be “tuned” to the specified nerve properties.

It is for the foregoing reason that the pulse generator 10 is provided with the means 12, 14 and 16 for varying the various current parameters of the pulse signal. The desired parameters are chosen by applying the electrodes 22 and 24 to the vagus nerve and varying the current parameters until the desired clinical effect is produced.

Since this “tuning” may have to be performed after the pulse generator is implanted, the present invention provides a means for varying the current parameters percutaneously. This is accomplished by a reed switch 30 associated with the implanted pulse generator 10 which is remotely controlled by electromagnet 32 and external programmer 34. The precise manner in which this is accomplished and the circuitry associated therewith is well known to those skilled in the art as the same technique has been widely used in connection with the “tuning” of cardiac pacemakers.

Even though a particular frequency or narrow band of frequencies is required for the desired purpose, it is believed that results may also be obtained by a variable frequency signal. If the frequency is varied by sweeping up and down by a random signal circuit or some other algorithm, there would be applied at least some of the time.

The device shown in FIG. 1 is intended for full implantation. It is also possible to practice the present invention with partial implantation. This is accomplished as shown in FIG. 2 by the use of a receiver 36 including a coil 38 and diode 40. The receiver is enclosed in an epoxy-titanium shell so that it can be implanted and is connected to the electrodes 22 and 24 on the vagus nerve through leads 18 and 20.

Located percutaneously is a pulse generator 42 which modulates the radio frequency transmitter 44 and delivers the radio frequency signal to antenna 46 which transmits the same to the receiver 36 when desired. It should be readily apparent that pulse generator 42 is also capable of being tuned so that the desired current parameters can be obtained. The pulse generator 42, transmitter 44 and antenna 46 could either be permanently worn on a person's body in the vicinity of the receiver 36 so that it need only be turned on when necessary or it may be separately carried in a person's pocket or the like and used whenever needed.

When the neurocybernetic prosthesis of the Zabara patents is utilized for preventing epileptic seizures, it can be utilized as described above wherein the current generator is turned only immediately preceding a convulsion. Many epileptics have sensory signs immediately preceding the convulsion called an aura. At the initiation of the aura, the patient will be able to turn on the device to prevent the seizure through the use of a manually operated switch. Even with a fully implanted prosthesis, a momentary contact switch, magnetically operated reed switch or a number of other devices could be provided which could be activated from outside of the body.

It is also possible to provide the prosthesis with a sensor-feedback system to block the seizure automatically. An example of such a system is shown in FIG. 3 and includes additional scalp electrodes 48 and 50 for measuring electroencephalographic waves. The output of the electrodes 48 and 50 is amplified by amplifier 52 and is then passed through filter 54 to level detector 56. When level detector 56 senses a significant and predetermined change in the electroencephalographic wave signal, it will automatically initiate the pulse generator 10 which will apply the required pulses to the electrodes 22 and 24 through runaway protection circuit 58 and voltage control circuit 60.

Although the sensing of electroencephalographic waves has been used above as an example for automatically turning on the neurocybernetic prosthesis, it should be apparent that other state parameters can be measured to provide a sensor-feedback system. Such other parameters might include respiration changes, heart rate changes, various auras or motor effects such as tics or myoclonic jerks. As a result, the prosthesis can be activated by sensor feedback producing a signal which precedes convulsive hypersynchronous discharge.

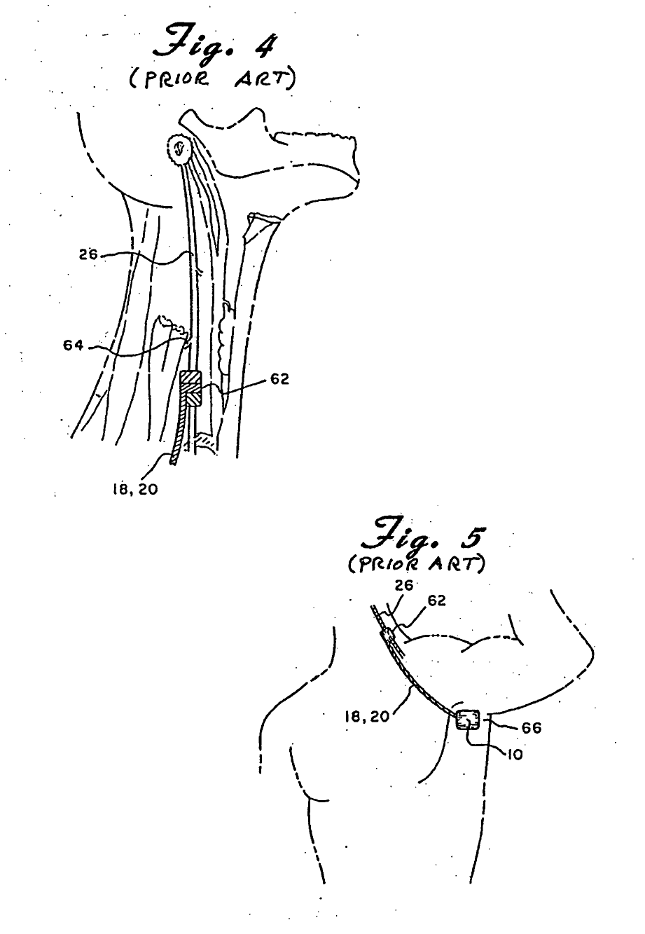

FIG. 4 illustrates the placement of the electrodes on the vagus nerve and shows the relationship of the vagus with adjacent structures. The electrodes are shown as a single electrode patch 62 which is known per se. Electrode patch 62 includes both the positive and negative electrodes.

Although it is theoretically possible to place the electrode patch 62 or separate electrodes substantially anywhere along the length of the vagus nerve 26, minimal slowing of the heart rate is achieved by placing the same below the inferior cardiac nerve 64. The electrodes may be placed on or adjacent to the vagus. It is preferred, however, that the negative electrode be proximal to the brain and the positive electrode may be used as an indifferent electrode and be placed in a different part of the body. For example, the case 26 of the implanted pulse generator 10 could, in some instances, be utilized as the positive electrode. It should be readily apparent to those skilled in the art that the terms “positive electrode” and “negative electrode” are merely relative; a positive electrode being one which is more positive than a negative electrode. Similarly, a negative electrode is one which is more negative than a positive electrode.

An electrode patch or cuff electrode such as that shown in FIG. 4 is the preferred embodiment. However, it should be readily apparent to those skilled in the art that various known electrodes such as a tripolar cuff electrode could be utilized. The electrodes may be placed either in direct contact with the nerve or in indirect contact with the neural tissue. There is no indication that placement of state of the art electrodes on the nerve itself would have a deleterious effect unless silver electrodes are utilized.

As shown in FIG. 5, the axilla or armpit 66 is the preferred location for placement of the pulse generator 10. The axilla provides protection for the pulse generator while allowing freedom of movement and is in proximity to the electrode patch 62. A subcutaneous tunnel between the incision made to implant the electrode patch and the incision made for implanting the pulse generator can be made with a metal rod. A plastic tube can then be inserted in the tunnel through which the electrode leads 18 and 20 can pass without excessive traction.

B. Improvement of the Present Invention

FIG. 6 shows an improved embodiment according to the present invention using a nerve conduction blocking electrode 100 positioned on the inferior cardiac nerve 64 such that the blocking electrode 100 is positioned between the heart and a stimulating electrode (i.e., electrode patch 62 of FIG. 4). Examples of electrode designs are shown in U.S. Pat. No. 4,979,511 to Terry, Jr. dated Dec. 25, 1990; U.S. Pat. No. 5,215,089 to Baker dated Jun. 1, 1993; U.S. Pat. No. 5,251,634 to Weinberg dated Oct. 12, 1993; U.S. Pat. No. 5,351,394 to Weinberg dated Oct. 4, 1994; U.S. Pat. No. 5,531,778 to Mashino dated Jul. 2, 1996; and U.S. Pat. No. 6,600,956 to Mashino dated Jul. 19, 2003 (all incorporated herein by reference).

The blocking electrode 100 is connected by a lead 102 to a controller (e.g., the pulse generator 10 of FIG. 1) adapted, in a preferred embodiment, to generate, at electrode 100, the blocking parameters that will be described hereafter. The blocking creates a neural block at the electrode 100. With such blocking parameters at blocking electrode 100, impulses from the_stimulating electrode are attenuated to avoid interference with the heart while the stimulating electrode 64 is stimulating the brain.

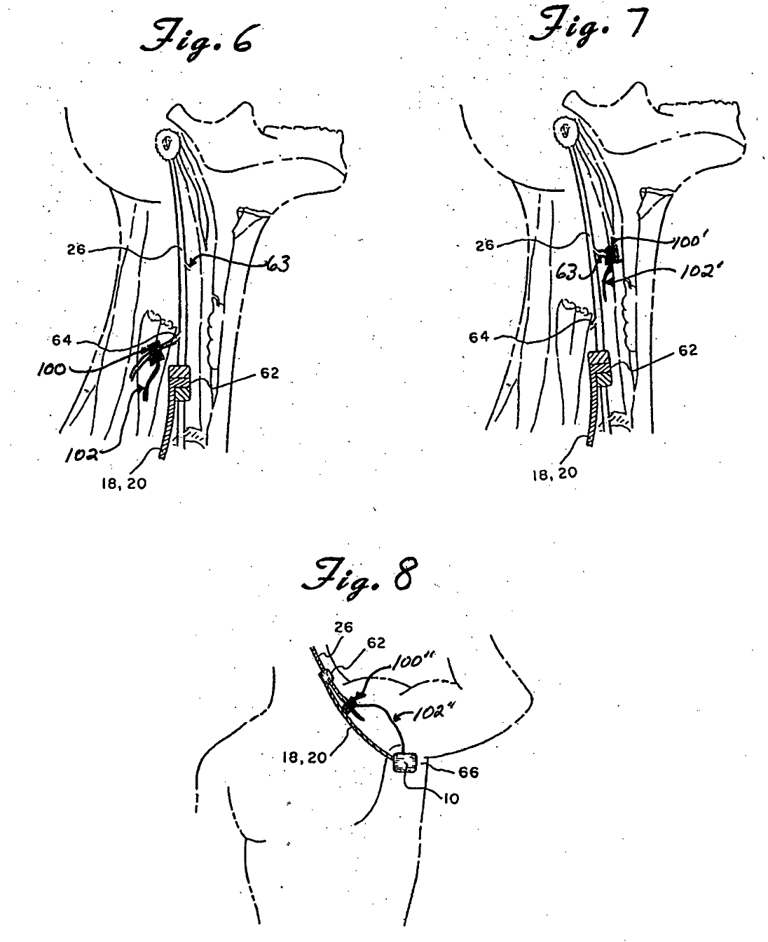

FIG. 7 shows an improved embodiment according to the present invention using a nerve conduction blocking electrode 100′ positioned on a nerve 63 innervating vocal cords (not shown). The electrode 100′ is energized by a signal on the conductor 102′ from the controller. The blocking electrode 100′ is positioned between the vocal cords and the stimulating electrode 62. In this embodiment, the blocking electrode 100′ blocks the nerve 63 to block signals from the stimulating electrode 62 to the vocal cords thereby reducing risks of adverse vocal effects during stimulation with the electrode 62.

FIG. 8 shows an improved embodiment according to the present invention using a nerve conduction blocking electrode 100″ positioned on the vagus nerve 26 distal to the stimulating electrode 62. The electrode 100″ is energized by a signal on the conductor 102″ from the controller. The blocking electrode 100′ is positioned between the organs of the cardiopulmonary system, gastrointestinal system and pancreobiliary system. In this embodiment, the blocking electrode 100′ blocks the vagus nerve distal to the stimulating electrode 62 to block signals from the stimulating electrode 62 to the organs of these systems thereby reducing risks of adverse vocal_effects during stimulation with the electrode 62.

A nerve block is, functionally speaking, a reversible vagotomy. Namely, application of the block at least partially prevents nerve transmission across the site of the block. Removal of the block restores normal nerve activity at the site. A block is any localized imposition of conditions that at least partially diminish transmission of impulses.

The vagal block of electrode 100, 100′, 100″ is desirable since unblocked pacing may result in afferent vagal and antidromic efferent signals having undesired effect on organs innervated directly or indirectly by the vagus (e.g., undesirable cardiac response or vocal response). Further, the afferent signals of the patch electrode 62 can result in a central nervous system response that tends to offset the benefits of the patch electrode 62 thereby reducing effectiveness of vagal stimulation.

The block may be intermittent and applied only when the vagus is stimulated by the patch electrode 62. The preferred nerve conduction block is an electronic block created by a signal at the vagus by an electrode 100 controlled by the previously described control system. The nerve conduction block can be any reversible block. For example, cryogenics (either chemically or electronically induced) or drug blocks can be used. An electronic cryogenic block may be a Peltier solid-state device which cools in response to a current and may be electrically controlled to regulate cooling. Drug blocks may include a pump-controlled subcutaneous drug delivery.

With such an electrode conduction block, the block parameters (signal type and timing) can be altered by a controller and can be coordinated with the pacing signals to block only during pacing._A representative blocking signal is a 500 Hz signal with other parameters (e.g., timing and current) matched to be the same as the pacing signal). The precise signal to achieve blocking may vary from patient to patient and nerve site. The precise parameters can be individually tuned to achieve neural transmission blocking at the blocking site.

While an alternating current blocking signal is described, a direct current (e.g., −70 mV DC) could be used. The foregoing specific examples of blocking signals are representative only. Other examples and ranges of blocking signals are described in the afore-mentioned literature (all incorporated herein by reference). As will be more fully described, the present invention gives a physician great latitude in selected stimulating and blocking parameters for individual patients.

As described, the parameters of the stimulating and blocking electrodes 62, 100 can be inputted via a controller and, thereby, modified by a physician. The blocking electrode can also be controlled by an implanted controller and feedback system. For example, physiologic parameters (e.g., heart rate, blood pressure, etc.) can be monitored. The blocking signal can be regulated by the controller to maintain measured parameters in a desired range. For example, blocking can be increased to maintain heart rate within a desired rate range during stimulation pacing.

With the benefit of blocking as described, the stimulation therapy can be applied more regularly (e.g., intermittently throughout the day) and need not be limited to times when an onset of need for therapy (e.g., a sensed onset of an epileptic seizure) is detected. This eliminates the need for complicated and potentially unreliable event detection and permits the use of the therapy to avoid an event before it starts.

C. Teachings of Prior Art for Near-Diaphragmatic Application

For ease of illustrating the present invention in a preferred embodiment for treating movement disorders, the description of the invention of U.S. Pat. No. 6,622,038 to Barret et al. dated Sep. 16, 2003 (the “038 patent” and incorporated herein by reference) is presented in this section of this application.

According to the '038 patent, a generally suitable form of neurostimulator for use in the apparatus and method of the invention of the '038 patent is disclosed, for example, in U.S. Pat. No. 5,154,172 (incorporated herein by reference) (the device also referred to from time to time herein as a NeuroCybernetic Prosthesis or NCP device (NCP is a trademark of Cyberonics, Inc. of Houston, Tex.)). Certain parameters of the electrical stimuli generated by the neurostimulator are programmable, preferably by means of an external programmer (not shown) in a conventional manner for implantable electrical medical devices.

Referring to FIG. 9, the neurostimulator (sometimes referred to herein as stimulus generator, signal generator, pulse generator, or simply the device), identified in the drawing by reference number 110 is implanted in a patient 112, preferably in the abdominal region, for example, via a left anterior thoracic or laporotomy incision just beneath the skin or outer dermal layer. For the preferred implementation and method of direct bilateral stimulation, lead-electrode pair 115, 116 is also implanted during the procedure, and the proximal end(s) of the lead(s) electrically connected to the neurostimulator. The lead-electrode may be of a standard bipolar lead nerve electrode type available from Cyberonics, Inc.

It will be understood that the overall device generally is required to be approved or sanctioned by government authority for marketing as a medical device implantable in a patient together with electrode means to treat the involuntary movement disorder by stimulation of a selected cranial nerve (e.g., the vagus nerve) of the patient. The treatment is performed using a predetermined sequence of electrical impulses generated by the pulse generator and applied to the selected cranial nerve at a location in a range, preferably, from about two to about three inches above or below the patient's diaphragm, for alleviating symptoms of the movement disorder in the patient.

The nerve electrodes 117, 118 are implanted on the right and left branches 119, 120, respectively, of the patient's vagus nerve at either a supra-diaphragmatic or sub-diaphragmatic location. The nerve electrodes are equipped with tethers for maintaining each electrode in place without undue stress on the coupling of the electrode onto the nerve itself. The location of this coupling is approximately two to three inches above or below the patient's diaphragm 122 for each branch 119, 120.

Neurostimulator 110 generates electrical stimuli in the form of electrical impulses according to a programmed regimen for bilateral stimulation of the right and left branches of the vagus. During the implant procedure, the physician checks the current level of the pulsed signal to ascertain that the current is adjusted to a magnitude at least slightly below the retching threshold of the patient. Typically, if this level is programmed to a value less than approximately 6 mA, the patient does not experience retching attributable to the vagus nerve stimulation (VNS) although variations may be observed from patient to patient. In any event, the maximum amplitude of the current should be adjusted accordingly until an absence of retching is observed, with a suitable safety margin. The retching threshold may change noticeably with time over a course of days after implantation, so the level should be checked especially in the first few days after implantation to determine whether any adjustment is necessary to maintain an effective regimen.

The bilateral stimulation regimen of the VNS preferably employs an intermittent pattern of a period in which a repeating series of pulses is generated for stimulating the nerve, followed by a period in which no pulses are generated. The on/off duty cycle of these alternating periods of stimulation and no stimulation preferably has a ratio in which the off time is approximately 1.8 to 6 times the length of the on time. Nominally, the width of each pulse is set to a value not greater than about 500 .mu.s, and the pulse repetition frequency is programmed to be in a range of about 20 to 30 Hz. The electrical and timing parameters of the stimulating signal used for VNS as described herein for the preferred embodiment of the '038 patent will be understood to be merely exemplary.

The intermittent aspect of the bilateral stimulation resides in applying the stimuli according to a prescribed duty cycle. The pulse signal is programmed to have a predetermined on-time in which a train or series of electrical pulses of preset parameters is applied to the vagus branches, followed by a predetermined off-time. Nevertheless, continuous application of the electrical pulse signal may also be effective in treating movement disorders.

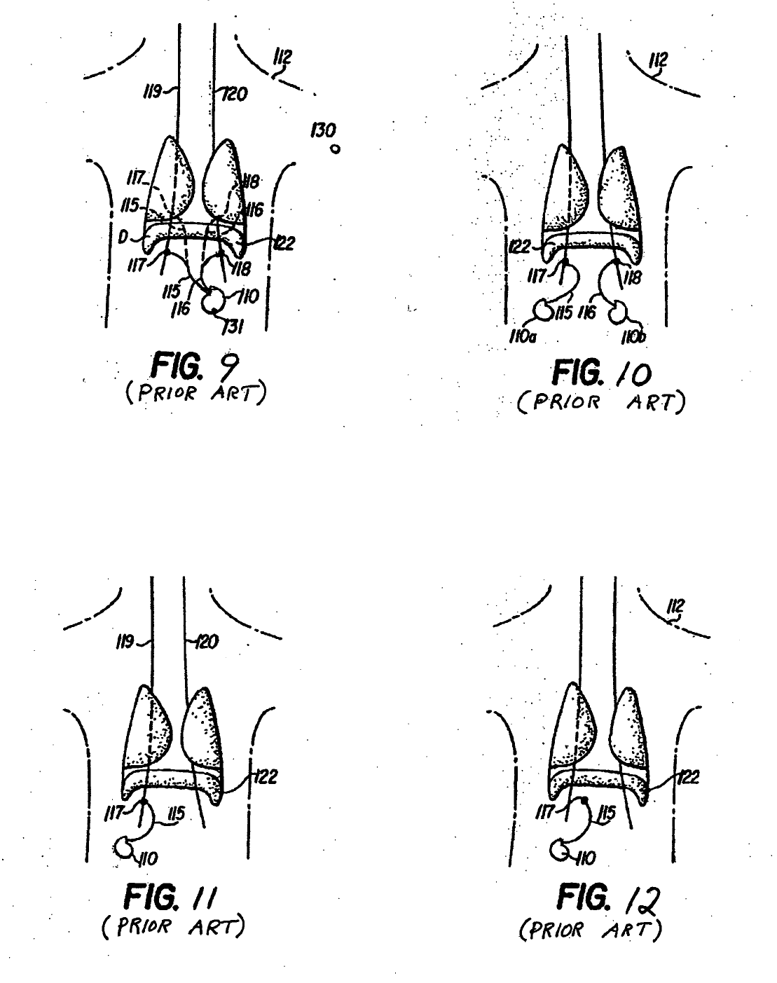

Also, as shown in FIG. 10, dual implanted NCP devices 110a and 110b may be used as the pulse generators, one supplying the right vagus and the other the left vagus to provide the bilateral stimulation. At least slightly different stimulation for each branch may be effective as well. Use of implanted stimulators for performing the method of the invention is preferred, but treatment may conceivably be administered using external stimulation equipment on an out-patient basis, albeit only somewhat less confining than complete hospitalization.

Implantation of one or more neurostimulators, of course, allows the patient to be completely ambulatory, so that normal daily routine activities including on the job performance is unaffected.

The desired stimulation of the patient's vagus nerve may also be achieved by performing unilateral supra-diaphragmatic or sub-diaphragmatic stimulation of either the left branch or the right branch of the vagus nerve, as shown in FIG. 11. A single neurostimulator 110 is implanted together with a lead 115 and associated nerve electrode 117. The nerve electrode 117 is implanted on either the right branch 119 or the left branch 120 of the nerve, preferably in a location in a range of from about two to about three inches above or below the patient's diaphragm 122. The electrical signal stimuli are the same as described above.

In a technique illustrated in FIG. 12, the signal stimuli are applied at a portion of the nervous system remote from the vagus nerve, for indirect stimulation of the vagus nerve in the vicinity of the diaphragmatic location. Here, at least one signal generator 110 is implanted together with one or more electrodes 117 subsequently operatively coupled to the generator via lead 115 for generating and applying the electrical signal internally to a portion of the patient's nervous system other than the vagus nerve, to provide indirect stimulation of the vagus nerve in the vicinity of the desired location. Alternatively, the electrical signal stimulus may be applied non-invasively to a portion of the patient's nervous system for indirect stimulation of the vagus nerve at the near-diaphragmatic location.

In treating the disorder, detection strategies such as sensing patient movement, particularly of the extremities, which appears to be random, uncoordinated and involuntary, may be employed to trigger the stimulation. To that end, a small accelerometer 130 in its own case may be separately implanted such as in a leg or arm of the patient to detect such movement. Or instead, the accelerometer may be mounted integrally in the same case that houses the neurostimulator. Alternatively, the vagal stimulation may be performed without need for detection of a symptom characteristic of the disorder or onset of the disorder. In that case, the stimulation is continuous, or it may be periodic, or intermittent during prescribed segments of the patient's circadian cycle. For example, stimulation may be periodic with a random frequency for the stimulating pulse waveform. In any event, this regimen of vagal stimulation is programmed into the neurostimulator device 110 (or 110a, 110b, as the case may be).

Since the patient is generally able to quickly recognize the symptoms of the movement disorder, where it is characterized by sudden onset or other random condition, provision may be made and preferably is made for patient activation of the neurostimulator for treatment of the particular movement disorder. For example, certain techniques of manual and automatic activation of implantable medical devices are disclosed in U.S. Pat. No. 5,304,206 to R. G. Baker, Jr. et al. (referred to herein as “the '206 patent”).

According to the '206 patent, means for manually activating or deactivating the stimulus generator may include a sensor such as a piezoelectric element 131 mounted to the inner surface of the generator case and adapted to detect light taps by the patient on the implant site. One or more taps applied in fast sequence to the skin above the location of the stimulus generator in the patient's body may be programmed into the device as the signal for activation of the generator, whereas two taps spaced apart by a slightly longer time gap is programmed as the signal for deactivation, for example. The therapy regimen performed by the implanted device(s) remains that which has been pre-programmed by means of the external programmer, according to the prescription of the patient's physician in concert with recommended programming techniques provided by the device manufacturer. In this way, the patient is given limited but convenient control over the device operation, to an extent which is determined by the program dictated and/or entered by the attending physician.

Where sense electrodes are to be utilized to detect onset of the movement disorder being treated, a signal analysis circuit is incorporated in the neurostimulator. Upon detection of the symptom of interest of the disorder being treated, the processed digital signal is supplied to a microprocessor in the neurostimulator device, to trigger application of the stimulating signal to the patient's vagus nerve.

The principles of the '038 patent may be applicable to selected cranial nerves other than the vagus nerve, to achieve the desired results.

D. Improvement of the Present Invention

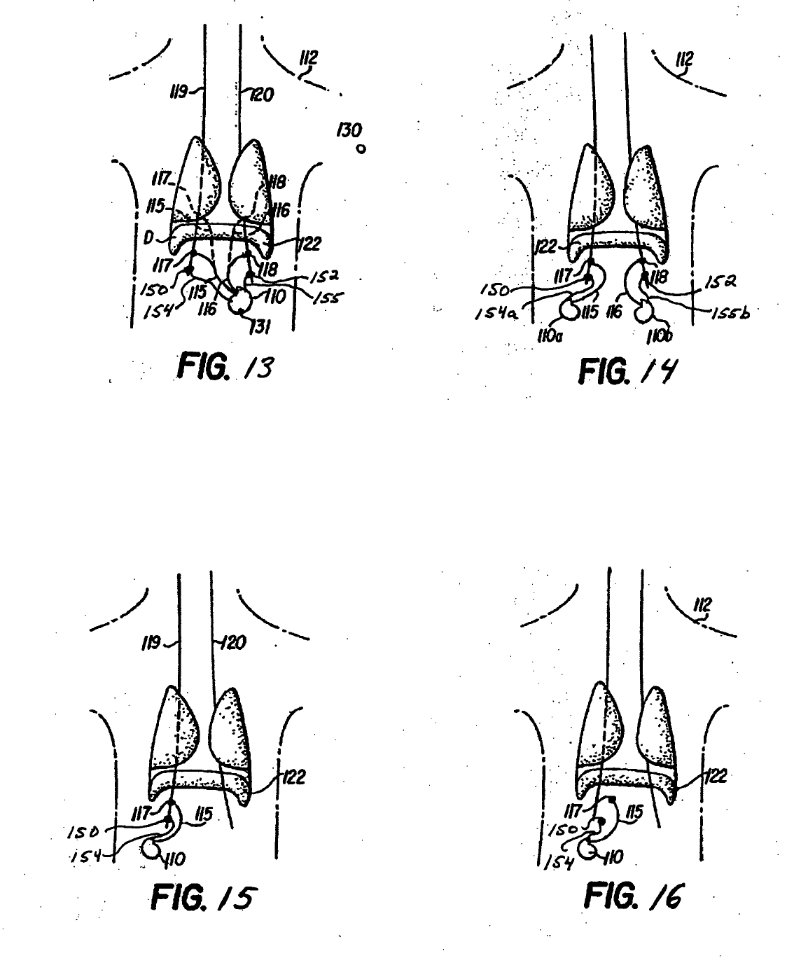

FIGS. 13-16 illustrate improvement of the prior art of FIGS. 9-12 with the addition of neural blocking electrodes. In FIG. 13, blocking electrodes 150, 152 are placed on nerves 19, 20 distal to the stimulating electrodes 117, 118. The blocking electrodes 150, 1523 are connected by leads 154, 155 to the controller 131 which, as well as generating the stimulation signal to electrodes 150, 152, generates a blocking signal. Similarly, in FIG. 14, blocking electrodes 150, 152 are placed distal to stimulation electrodes 117, 118 and connected to respective generators 10a, 10b by leads 154a, 155b. The generators generate blocking signals to electrodes 150, 152 as well as stimulating signals to electrodes 117, 118. In FIG. 15, a single blocking electrode 150 is on nerve 119 distal to stimulating electrode 117 and connected to generator 110 by lead 154 to receive a blocking signal. In FIG. 16, the blocking electrode is indirectly coupled to the nerve 117 distal to the indirect coupling of the stimulation electrode 115. The electrode 150 is connected to generator 110 by lead 154 to receive a blocking signal. In all of the above, the blocking signal is as previously described.

In the above embodiments, the distal connection of the blocking electrodes results in a blocking signal to at least partially block distal flow of stimulation signals past the blocking site. This reduces_adverse side effects to gastro-intestinal and pancreobiliary organs which would result from unblocked signals.

With the foregoing detailed description of the present invention, it has been shown how the objects of the invention have been attained in a preferred manner. Modifications and equivalents of disclosed concepts such as those which might readily occur to one skilled in the art, are intended to be included in the scope of the claims which are appended hereto.

1. A method of controlling or preventing involuntary movements such as caused by epileptic seizures, cerebral palsy, Parkinson's disease, spasticity, motor disorders and the like comprising:
applying a stimulation electrical signal to the vagus nerve to thereby prevent or control such movement
applying a neural conduction block to a vagus nerve of said patient at a blocking site with said neural conduction block selected to at least partially block nerve impulses on said vagus nerve at said blocking site.

2. A method according to claim 1 wherein said neural conduction block is applied to said nerve between a location of application of said stimulation electrical signal and an organ to be shielded from adverse effects of said stimulation electrical signal.

3. A method according to claim 1 wherein said neural conduction block is applied during application of said stimulation electrical signal.

4. A method according to claim 1 wherein application of said neural conduction block is variable by a controller to alter a characteristic of said block.

5. A method according to claim 1 wherein said neural conduction block is a cryogenic block

6. A method according to claim 1 wherein said neural conduction block is a pharmocologic block

7. A method according to claim 1 wherein said neural conduction block is an electrical conductive block

8. A method according to claim 1 further comprising determining that an involuntary movement is going to occur and thereafter applying said pulsed electrical signal to said vagus nerve.

9. An apparatus for controlling or preventing involuntary movements such as caused by epileptic seizures, cerebral palsy, Parkinson's disease, spasticity, motor disorders and the like comprising:
a stimulation electrical signal generator capable of generating pulses having a frequency of approximately between 30 and 300 cycles per second with each pulse having a duration of between approximately 0.3 and 1 millisecond;
a positive electrode adapted to be applied to a person's body and means electrically connecting said electrode to said pulse generator;
a negative electrode adapted to be applied to a person's body adjacent the vagus nerve;
means for electrically connecting said electrode to said generator;
an electrically controllable neural conduction electrode adapted to be placed on a vagus nerve of said patient at a blocking site between a location of application of said stimulation electrical signal and an organ to be shielded from adverse effects of said stimulation electrical signal; and
a blocking signal generator for generating a blocking signal selected to at least partially block nerve impulses on said vagus nerve at said blocking site.

10. A method of controlling or preventing involuntary movements such as caused by epileptic seizures, cerebral palsy, Parkinson's disease, spasticity, motor disorders and the like comprising:
determining that an involuntary movement is going to occur and thereafter applying a stimulation electrical signal to the vagus nerve as a point below the inferior cardiac nerve to thereby prevent or control such movement; and
applying a neural conduction block to a vagus nerve of said patient at a blocking site with said neural conduction block selected to at least partially block nerve impulses on said vagus nerve at said blocking site.

11. A method according to claim 10 wherein said neural conduction block is applied to said inferior cardiac nerve.

12. A method according to claim 10 wherein said neural conduction block is applied during application of said stimulation electrical signal.

13. A method according to claim 10 wherein application of said neural conduction block is variable by a controller to alter a characteristic of said block.

14. A method according to claim 10 wherein said neural conduction block is a cryogenic block.

15. A method according to claim 10 wherein said neural conduction block is a pharmocologic block.

16. A method according to claim 10 wherein said neural conduction block is an electrical conductive block.

17. A method of treating patients suffering from a movement disorder, which comprises the step of:
stimulating a patient's vagus nerve with an electrical pulse signal applied directly or indirectly thereto at a location in the immediate vicinity of the patient's diaphragm, including selectively programming electrical and timing parameters of said electrical pulse signal according to a predetermined therapy regimen for alleviating the disorder, and
applying a neural conduction block to said vagus nerve of said patient at a blocking site with said neural conduction block selected to at least partially block nerve impulses on said vagus nerve at said blocking site.

18. A method according to claim 17 wherein said neural conduction block is applied to said nerve between a location of application of said stimulation electrical signal and an organ to be shielded from adverse effects of said stimulation electrical signal.

19. A method according to claim 17 wherein said neural conduction block is applied during application of said stimulation electrical signal.

20. A method according to claim 1 wherein application of said neural conduction block is variable by a controller to alter a characteristic of said block.

21. A method according to claim 1 wherein said neural conduction block is a cryogenic block.

22. A method according to claim 1 wherein said neural conduction block is a pharmocologic block.

23. A method according to claim 1 wherein said neural conduction block is an electrical conductive block.

24. A method of treating patients suffering from involuntary movement disorders by stimulating a selected cranial nerve of the patient with an electrical signal applied to induce a signal up the nerve toward the brain from a location in the vicinity of the patient's diaphragm, including programming electrical and timing parameters of said electrical signal to ameliorate said disorder and programming electrical and timing parameters of a neural_conduction block selected to at least partially block nerve impulses on said nerve at a blocking site.
